(19) United States
(12) Patent Application Publication
(10) Pub. No.: US 2018/0052965 A1
(43) Pub. Date: Feb. 22, 2018

(54) Context setting algorithmic process for care companion device, self sensing and bridging a care pod to the care giver for non intrusive, seamless, continuous care monitoring for improving quality of care experience for care receiver

(71) Applicant: magpie 360 Inc., Andover, MA (US)

(72) Inventors: Ramesh Tumkur, Westford, MA (US); Kannan Ramasamy, Andover, MA (US)

(21) Appl. No.: 15/079,912

(22) Filed: Mar. 24, 2016

Related U.S. Application Data
(60) Provisional application No. 62/178,590, filed on Apr. 15, 2015.

(51) Int. Cl. G06F 19/00; A61B 5/00; G06F 21/60

(52) U.S. Cl. CPC G06F 19/3418; G06F 21/602; A61B 5/0008; A61B 5/0022

(57) ABSTRACT

A “care pod” is a virtual container that encompasses a care receiver and devices such as biosensors and a medical kit that form the sensors ecosystem. The care pod is contextually and algorithmically nested and locked to a “care companion device”. The “care companion device” has the intelligence to self-sense and connects the “care pod” to the “care giver”. The logistics and positioning of the caregiver thus becomes immaterial; be it onsite or remote, the caregiver is continually, on demand, and non-intrusively able to access the current and historical state of the care receiver in a secure manner. Caregivers are able to monitor care receivers in real time through meaningful alerts and collaborate to drive care outcomes.

### RELATED APPLICATIONS

This application claims priority from U.S. Provisional Patent Application Ser. No. 62/178,590 filed on Apr. 15, 2015.

### BACKGROUND OF THE INVENTION

“Know thy patient (care receiver)” at all times is an imperative for a care giver. A “care pod” is a virtual container that encompasses a care receiver and devices such as biosensors and a medical kit that form the sensors ecosystem. The care pod is contextually and algorithmically nested and locked to a “care companion device”. The care companion device has the intelligence to self-sense and connects the care pod to the “care giver”. The logistics and positioning of the caregiver thus becomes immaterial; be it onsite or remote, the caregiver is continually, on demand, and non-intrusively able to access the current and historical state of the care receiver in a secure manner. Caregivers are able to monitor care receivers in real time through meaningful alerts and collaborate to drive care outcomes.

### SUMMARY

The fully functioning framework of a containerized care platform is illustrated in FIG. 1 with all the components of a care pod, a care companion device and a care cloud with mobile apps.

Central to the containerized care is the care receiver. The containerized care is illustrated in FIG. 2.

The care receiver is the “human intranet” that is so complex and yet structured into containerized micro services encompassing nervous system, digestive system, skeletal system and others, each in turn with supported organs constantly in communication that is simply amazing to learn about and yet mind boggling to decipher. The human intranet continually generates analog data signals that when accessed can provide valuable data on its state and the well-being of the body and the processes they support measured through the data signals received in the form of vitals such as blood pressure, heart rate, oxygen saturation rate, weight, activity level, sugar levels, etc.

From a care receiver perspective such signals when untapped end up becoming dark data and take away from the quality of care. Tapping into the dark data can positively impact quality of care, pro-active lifesaving interventions, valuable insights and reduced operational and cost overheads of care. Over a secure fork-lifted white-list from the care cloud, a care companion device intelligently becomes aware of the care pod and key pairs as the care pod becomes active for transmission of data to the care cloud over an automated non-intrusive process. A care pod is a virtualized container surrounding the caregiver with listening instrumentation, environmental sensors, a biosensor patch, and others to tap into the analog data. The contextual locking arrangement thus voids any calls for care receiver to intervene with the care pod environment either with pairing of keys, battery check or other reactive measures. The care pod is managed and proactively monitored remotely void of any human or physical intervention.

The care companion device itself is void of any visible indicators or alarms to obviate the need for interaction with the care receiver. With this approach the care pod environment processes, including but not limited to key pair process, checking battery status, monitoring the status of the care pod, are known beforehand and proactively managed at the care cloud through automated processes.

The care companion device is the non-intrusive, plug and powered device that is algorithmically and contextually aware of its care pod. Using an algorithmically self-sensing process it connects and harnesses the care receiver analog signals emanating within the care pod realm into events for secure transport to the care cloud. The upstream care cloud normalizes, correlates, and processes the transported real-time events to provide actionable alerts and insights.

### BRIEF DESCRIPTION OF THE DRAWINGS

FIG. 1 illustrates the fully functioning framework of a containerized care platform.

FIG. 2 illustrates the containerized care structure.

FIG. 3 illustrates the sequence of algorithmic steps linking the care cloud and care capsule.

FIG. 4 illustrates the remote capsule based key-pair process initiated from the care cloud void of care receiver intervention.

FIG. 5 illustrates the care receiver UUID.

FIG. 6 illustrates the ‘care receiver’ proximity.

FIG. 7 illustrates a snapshot of a care receiver vitals focused dashboard.

FIG. 8 illustrates the care companion device with an automated contextual awareness process.

FIG. 9 depicts a multiple care companion device environment co-existing without being aware of other care pods within their vicinity yet focused on their assigned environment, giving the platform a multi-tenant visibility.

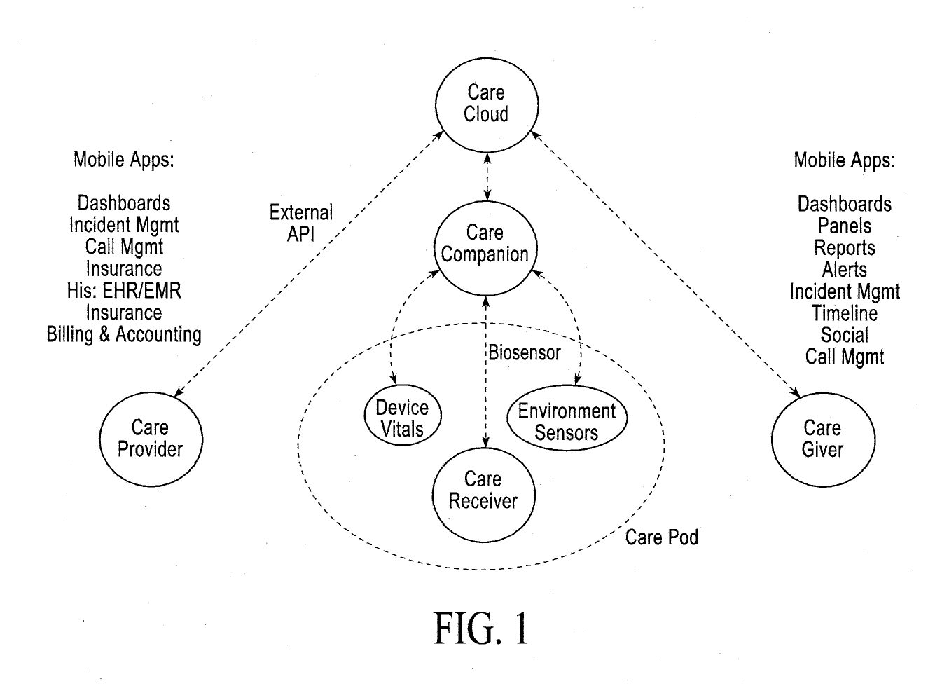

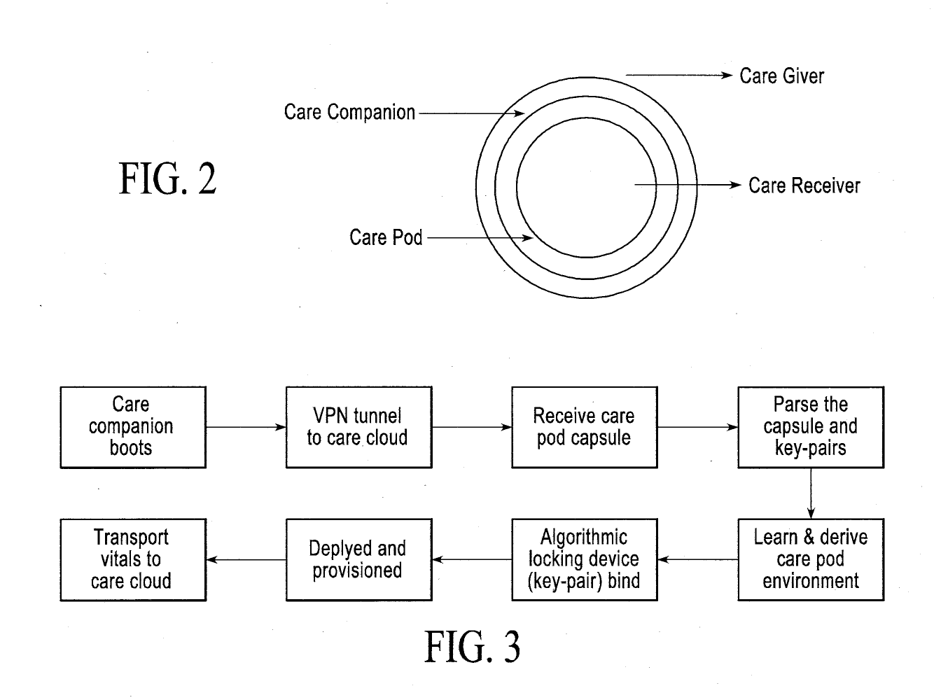

### DETAILED DESCRIPTION

Our invention is a “care companion device” that algorithmically and contextually self senses and locks to a care pod. The device supports network ports configured to receive dark data radio signals, a plurality of radio receivers for data reception, security filters to remove noise from signals, a locking module to ascertain and protect the distinct pod, an automated remote key-pairing process that detects and pairs the devices making up the care pod context, a processing container for normalization and correlation for contextual awareness, a memory unit for recording, a security transporter for transporting real time processed data over a secure management tunnel to the upstream cloud for analytics, actionable alerts and reports around care receiver ‘vitals’.

As a part of the plug and power automated provisioning process, the device boots up when powered, ascertains its next step and docks to the care cloud over a secure VPN session for assessment of its state, receives upon security validation its “care capsule”, and becomes contextually aware of its care pod. A “care capsule” is a dictionary object that encompasses the custom attributes and contextual attributes generated in the cloud that are specific to a distinct care companion device. It gives the care companion device the “sense” of its environment and defines its purpose. With the care capsule, the care companion device becomes aware of the “care receiver” for monitoring and care. The sequence of algorithmic steps is illustrated in FIG. 3.

With the capsule security data, which is a dictionary of white-list elements that make up the care pod, the parsing process begins by validating and extracting the keys and values in the dictionary. The key-value pairs dictate the white-listed environment that makes the care pod.

The capsule is crucial for the care companion device to customize and ascertain its environment and thus become contextually aware of it. Once validated and parsed it goes into stealth mode and sniffs for the care pod elements, namely biosensors and medical devices to show up and launch the key-pairing process. Once the biosensors and medical devices making up the white-list are ascertained, it initiates the pairing process, effectively locking to the devices as belonging to its care pod.

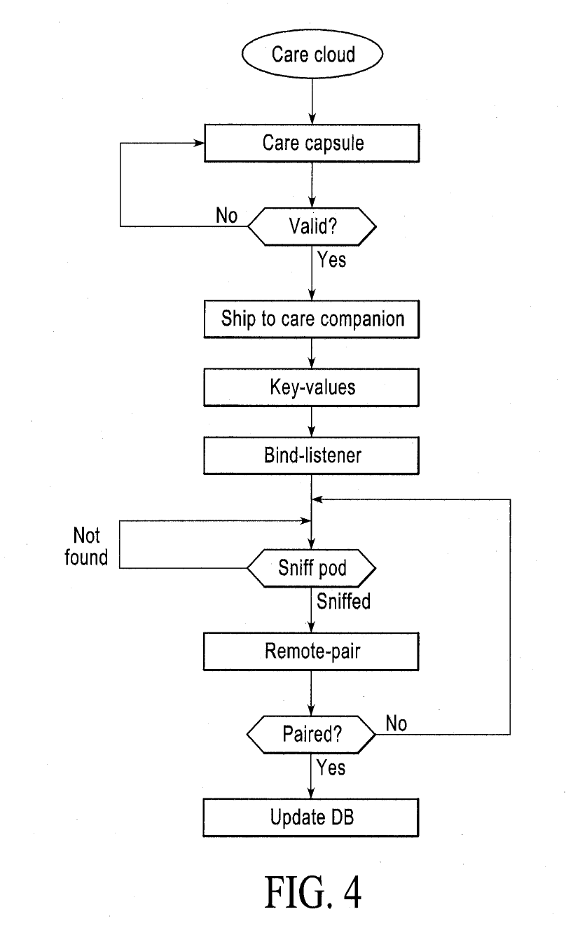

The care companion device then begins the process of sensing the care pod and care receiver for the data and vitals. The captured real time events encompassing vitals and sensor data are transported to the upstream care cloud for indexing, analytics and actionable alerts. FIG. 4 illustrates the remote capsule based key-pair process initiated from the care cloud void of care receiver intervention.

Caregivers can access data to “know thy patient (care receiver)” from anywhere, anytime and anyplace using the mobility feature of the platform. A graphical interface implemented and supported on iPhone and Android apps enables the caregiver to access, analyze, decide and act directly or through other agents who are a part of the caregiver network.

The “care giver” dashboard is linked to a unique secure and protected care receiver ID illustrates the patient pod (care pod), instrumentation, analytics and services. The care pod is represented as a dashboard panel on the care giver mobile app, providing the geo location, biosensors, instruments, and environmental assets mapped to the distinct care receiver ID.

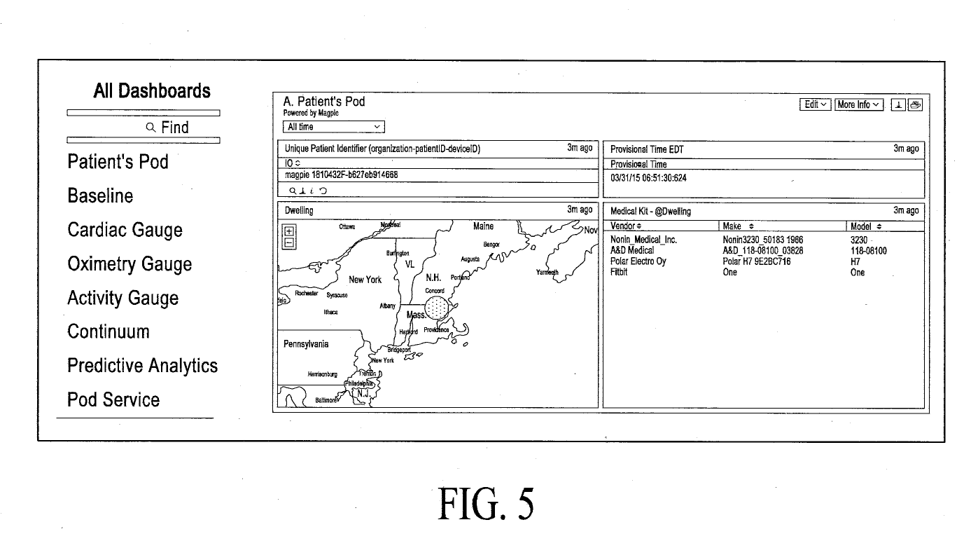

The care receiver UUID as illustrated in FIG. 5 is made up of 3 fields, namely the organization ID relating to the care provider, patient identifier generated by the care console, and the care companion device ID. This UUID is unique across any care receiver deployed instance, protecting privacy of the patient by restricting the personal record to the provider entity.

The provider entity is the single source authorized to map patient UUID to patient private attributes.

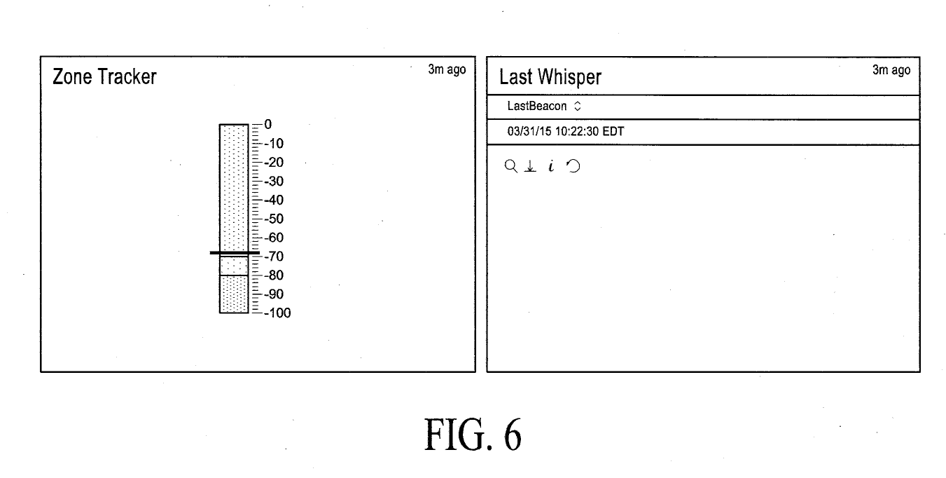

The dashboard in FIG. 6 illustrates the care receiver proximity.

Other instrumentation panels offer vital readings over various stages providing real time, continuous and historical data related to the care pod.

This is valuable for monitoring and auditing overall effectiveness of care delivery and facilitating the process of reimbursement to the care provider. The care companion device itself is monitored as it is the glue that enables the care pod to perform its duties for the care receiver to meaningfully connect to the care giver.

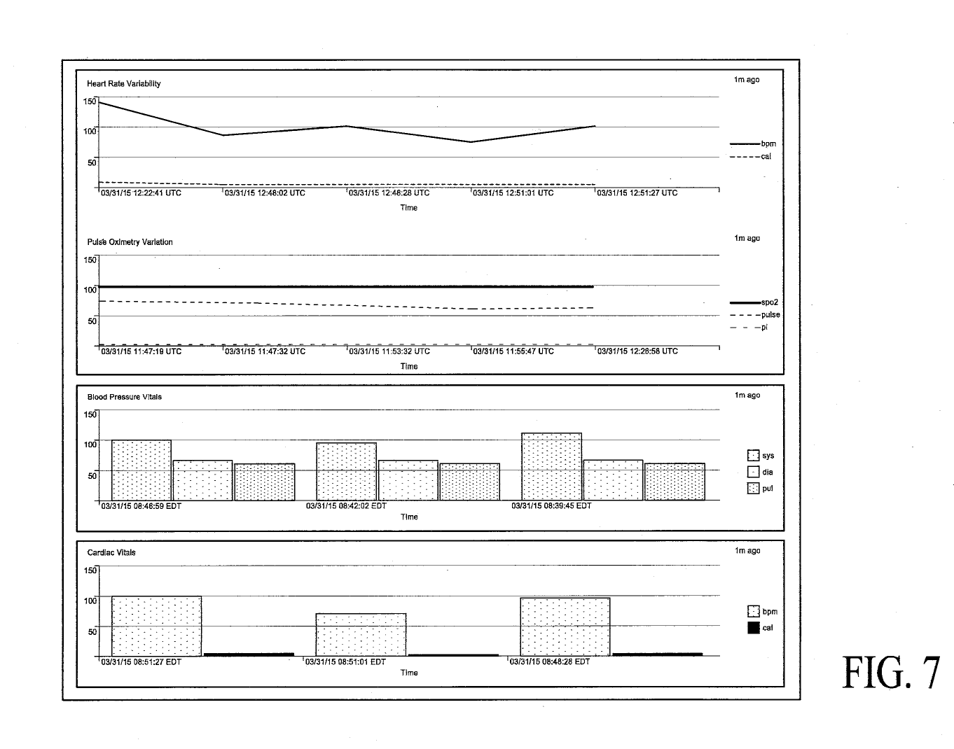

A snapshot of a care receiver vitals focused dashboard is illustrated in FIG. 7 for completeness. The dashboards are focused around disease states as well as distinct vitals of interest associated to those disease states.

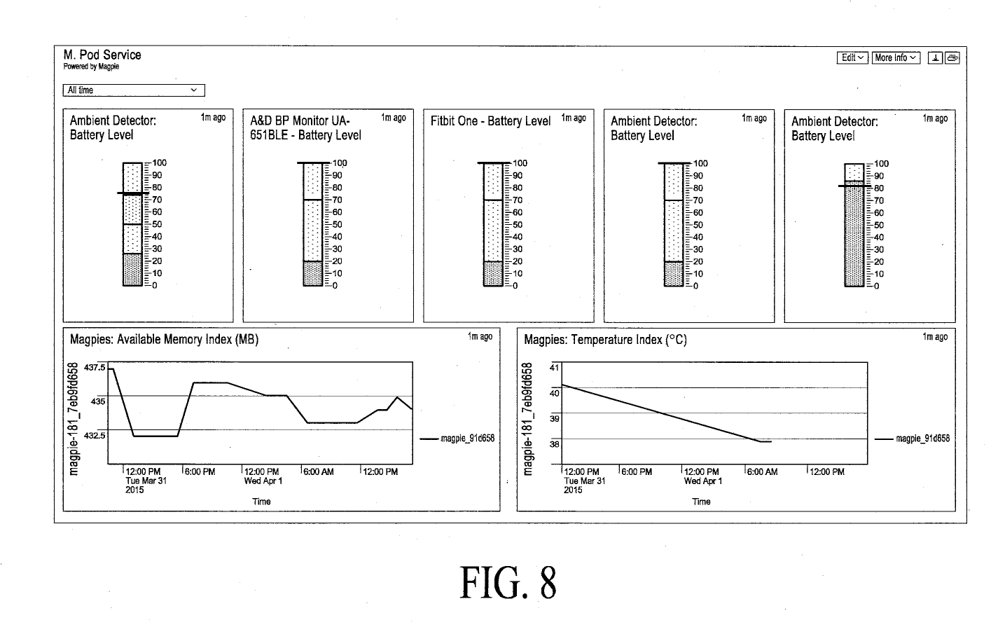

Thus, care companion device upkeep and uptime is integrated into the process without user intervention. The key pair process, docking the care pod, ascertaining the white list that makes up the care pod are all illustrative of this automated contextual awareness process. This is illustrated in FIG. 8.

The data from the care pod and containerized care platform are transported to electronic health record (EHR) systems electronically to make this information actionable within the current medical system accessed by the caregiver.

The net benefit is an automated and paperless experience with one single source of truth at EHR.

This helps with care receiver diagnosis, personal history, and collaboration among multiple care givers at distinct times using the alerts and notifications provided by the containerized companion platform to the medical systems, or optionally on the care giver's mobile device.

### Multiple Care Companion Device and Contextual Lock

The care companion device and the care cloud in sum total form a secure data center designed to stay on-line, containerized, fully-operational and designed to protect privacy of data transiting end-to-end. Each care companion device is locked to its care pod due to the distinct white-listed attributes within the care pod delivered care capsule. Such a contextual locking arrangement prevents one care companion device from listening to or cross-talking with neighboring care pods avoiding cross-pollination of data.

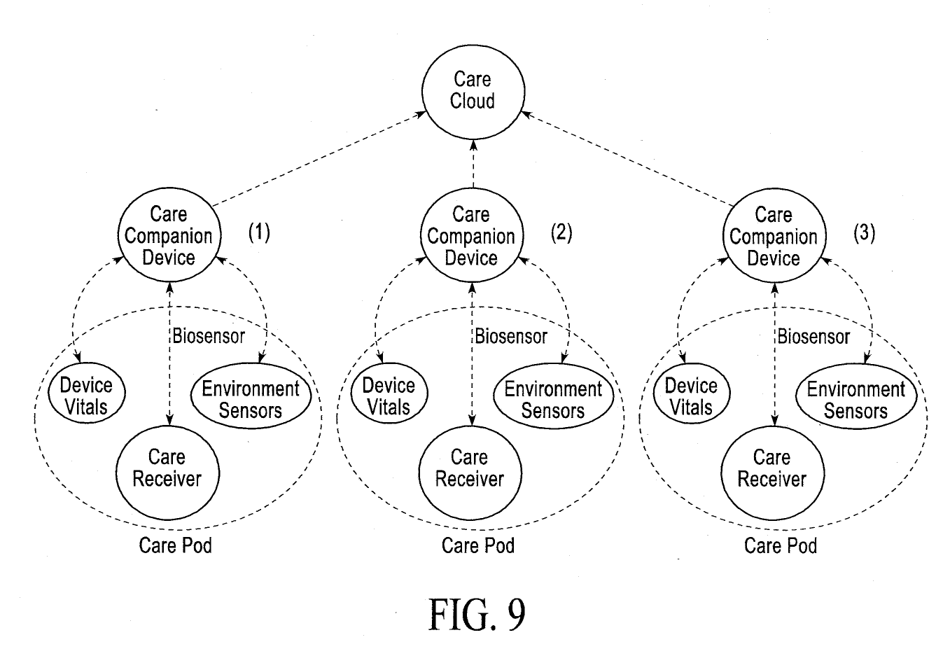

FIG. 9 depicts a multiple care companion device environment co-existing without being aware of other care pods within their vicinity yet focused on their assigned environment. It is the care cloud that gives the platform a multi-tenant visibility.

On the device security front, the care companion device has the vitals recorded in a memory based file system over a battery backup facility. Opening the device or battery causes the vitals to be wiped out. There are no ingress physical ports other than a power port. The only outbound network port is to the care cloud over mutually authenticated TLS session. The care cloud is aware of the state of the care companion device from event flow and time perspective, thus enabling real time security monitoring.

The invention has been described in terms of particular embodiments. Other embodiments are within the scope of the following claims. For example, the steps of the invention can be performed in a different order and still achieve desirable results.

What is claimed is:

1. A system comprising a care companion device including a USB powered credit card sized device and a care pod, wherein the care companion device virtually locks, manages and monitors a care pod, wherein the care pod includes an eco-system of biosensors, medical devices, and bio patch that are radio powered with a care giver focus whose status is known and managed remotely by the care companion device at all times.

2. The system of claim 1, wherein the care pod is distinct and known to care companion device and built-in security mechanisms prevent other care receivers or other devices not part of the eco-system from cross talking or interfacing with the care pod environment thereby providing a dedicated and virtual care companion device.

3. The system of claim 1, wherein a plurality of sensors in the care pod comprising a care receiver ecosystem are always locked to the care companion device.

4. The system of claim 1, wherein the care companion device has back-up battery power to manage and monitor the care pod.

5. The system of claim 1, wherein the care companion device is docked to the care cloud over multiple distinct encrypted communication channels across geographical locations for redundancy to ensure the real-time events are transported to a care cloud.

6. The system of claim 1, wherein the care companion device has a built-in mechanism to switch to multiple or back-up transport networks during outages.

7. The system of claim 1, wherein the care companion device is provisioned and deployed from a care cloud with no intervention at the point of care and without care receiver interaction, and a care capsule injected from the care cloud gives the care companion device a contextual awareness of the care pod over an automated process void of any point of care involvement.

8. The system of claim 1, further comprising a remote key pairing processes wherein the care companion device by use of a care capsule ascertains its care pod and remote key pairs with the care pod environment and associated devices, whereby the remote key pairing over machine-to-machine communication voids any physical intervention or point of care intervention.
